(19) United States
(12) Patent Application Publication
(10) Pub. No.: US 2016/0040061 A1
(43) Pub. Date: Feb. 11, 2016

(54) FLEXIBLE POLYMERIC MATERIALS CONTAINING TRIBOLUMINESCENT COMPOUNDS, PROTECTIVE DEVICES CONTAINING SUCH MATERIALS, AND METHODS OF MANUFACTURING THE SAME

(71) Applicant: FULLPROOF, LLC, Tampa, FL (US)

(72) Inventors: Elizabeth KROWNE, Washington, DC (US); Clifford KROWNE, Alexandria, VA (US); Christopher Todd MARKS, Tampa, FL (US); Natalie KHAWAM, Tampa, FL (US)

(21) Appl. No.: 14/773,601

(22) PCT Filed: Mar. 7, 2014
(86) PCT No.: PCT/US2014/021577

§ 371 Date: Sep. 8, 2015

Related U.S. Application Data
(60) Provisional application No. 61/775,452, filed on Mar. 8, 2013.
(60) Provisional application No. 61/775,458, filed on Mar. 8, 2013.
(60) Provisional application No. 61/775,437, filed on Mar. 8, 2013.

(51) Int. Cl. C09K 11/06; B29C 41/02; B29C 41/00; A41D 19/00; A61F 6/04

(52) U.S. Cl. CPC C09K 11/06; A41D 19/0082; A61F 6/04; B29C 41/003; B29C 41/02; C09K 2211/182; B29K 2105/0005

(57) ABSTRACT

Protection devices embedded with triboluminescent compounds can, through light emissions, change color in response to mechanical injury, alerting users to the injury.

### CROSS REFERENCE TO RELATED APPLICATIONS

This application claims priority to U.S. Provisional Patent Application Nos. 61/775,437, filed Mar. 8, 2013, 61/775,452, filed Mar. 8, 2013, and 61/775,458, filed Mar. 8, 2013, each of which is incorporated herein by reference in its entirety.

### BACKGROUND OF THE INVENTION

1. Field of the Invention

The invention relates to the use of triboluminescent compounds in materials used in making protective devices, such as condoms, gloves, and food packaging.

2. Brief Description of Related Art

Flexible polymeric materials are frequently used to provide physical barriers in a number of contexts.

Condoms, for example, are frequently used to prevent pregnancy and to help stop the transmission of sexually transmitted diseases (STDs) such as HIV. Condoms are most commonly made of latex or polyurethane, but have also been made of other materials, such as polyisoprene and lamb intestine. If used properly, a condom is very effective at reducing the risk of HIV infection during sexual intercourse. Using a condom also provides protection against other sexually transmitted diseases and protection against pregnancy. In the laboratory, latex condoms are very effective at blocking transmission of HIV because the pores in latex condoms are too small to allow the virus to pass through. However, outside of the laboratory condoms are less effective because people do not always use condoms properly.

Latex gloves are one of the most important supplies that are used by industries throughout the world. Whether it be a nurse in a hospital or a cook in a cafeteria, latex gloves help keep their wearers—and others they come in contact with—clean and safe from infections and other substances that could have negative consequences.

Plastic films are frequently used as to hermetically seal packaging for a variety of items, such as electronic instruments, especially those which may be subject to damage if scratched or immersed in a liquid, jewelry, medical products, whether instruments or medicines, and food products, to name a few.

Packaging for food requires protection, tampering resistance, and special physical, chemical, or biological needs. The general principle of food packaging is better containment, protection against physical, chemical, biological and environmental factors. It is to aid consumers in using products and to communicate to and educate consumers about the ingredients, nutritional contents and the materials used to provide the protection.

In each of the foregoing applications, mechanical integrity of the flexible polymeric material is of paramount importance.

A large body of research in the United States has shown that rates of breakage, caused by fault in the condom itself, are less than 2 condoms out of every 100 condoms. Presently, latex condoms are tested for holes with an electrical current. If the condom passes, it is rolled and packaged. In addition, a portion of each batch of condoms is subject to water leak and air burst testing. Such testing methods cannot test for any damage that occurs—whether intentional or not—after point of sale. Additionally, a failure rate of just under 2% does not account for purposeful or intentional failure. Purposeful or intentional damage to a condom's mechanical integrity may occur when unbeknownst to one of the partners, the other partner has compromised the condom's integrity by damaging it in some way, probably not detectable to the naked eye. Such intentional damage may be due to one of the partners wishing to intentionally infect his or her partner with a disease or to inseminate without one partner's knowledge, in the hopes of achieving pregnancy.

There is little question that failure of surgical or examination gloves in the medical field and related fields can be a serious issue. Other related fields where this can occur include biotechnology, nanoscience, chemistry, and material science. Another related area to medicine is the veterinary field, from research to clinics. Finally, there is the food industry. Failure during usage can simply result from the mechanical stresses and chemical exposures the glove meets, causing a rupture at some point in the material. Failure may be as insignificant as a pinhole, or a line rupture producing a tear with a definite width and length. It is important to realize that even a pinhole can leak significant amounts of fluid onto a hand, given the pressures encountered during medical exams or other usage, like in a cafeteria.

Failure prior to usage, assuming improper handling by the user(s) is not a cause, can occur during glove manufacture. This rate of failure could be as high as a few percent. Most likely failure of the glove is due to manufacturing causes. However, purposeful failure may occur when unbeknownst to the user(s), there may be malicious intent from parties not wanting examinations to succeed including thwarting criminal and civil investigations. Such damage to the glove would probably not detectable with the naked eye. Most likely insults to the mechanism and physical integrity of the glove can occur with pinhole punctures. Of course, with constant threats of terror, from individuals or groups wishing to encourage the spread of germs by compromising gloves used in the food industry, one could expect this to be another important concern.

There are several functions of food packaging, including:
Physical protection—The food enclosed in the package may require protection from, among other things, shock, vibration, compression, temperature, etc.
Barrier protection—A barrier from oxygen, water vapor, dust, etc., is often required. Permeation is a critical factor in design. Some packages contain desiccants or Oxygen absorbers to help extend shelf life. Modified atmospheres or controlled atmospheres are also maintained in some food packages. Keeping the contents clean, fresh, and safe for the intended shelf life is a primary function.
Containment or agglomeration—Small items are typically grouped together in one package for reasons of efficiency. Powders and granular materials need containment.
Information transmission—Packages and labels communicate how to use, transport, recycle, or dispose of the package or product. Some types of information are required by governments.
Marketing—The packaging and labels can be used by marketers to encourage potential buyers to purchase the product. Package design has been an important and constantly evolving phenomenon for several decades. Marketing communications and graphic design are applied to the surface of the package and (in many cases) the point of sale display.
Security—Packaging can play an important role in reducing the security risks of shipment. Packages can be made with improved tamper resistance to deter tampering and also can have tamper-evident features to help indicate tampering. Packages can be engineered to help reduce the risks of package pilferage: Some package constructions are more resistant to pilferage and
   some have pilfer indicating seals. Packages may include authentication seals to help indicate that the package and contents are not counterfeit. Packages also can include anti-theft devices, such as dye-packs, RFID tags, or electronic article surveillance tags, that can be activated or detected by devices at exit points and require specialized tools to deactivate. Using packaging in this way is a means of retail loss prevention.
Convenience—Packages can have features which add convenience in distribution, handling, stacking, display, sale, opening, reclosing, use, and reuse.
Portion control—Single serving packaging has a precise amount of contents to control usage. Bulk commodities (such as salt) can be divided into packages that arc a more suitable size for individual households. It also aids the control of inventory: selling sealed one-liter-bottles of milk, rather than having people bring their own bottles to fill themselves.

An insult to the mechanical integrity of protective devices such as condoms, gloves, and food packaging can have numerous undesirable consequences, such as unwanted pregnancies, spread of communicable diseases (including STDs), contaminated foods, and food spillages. One deficiency in flexible polymeric materials used in these types of products is that there is a lack of simple means for the unskilled user to evaluate the integrity of the product. That is, the observer is left to his or her devices to detect a line rupture, pinhole rupture, or any other type of rupture. For small ruptures, this may be a problem, because the naked eye has limited resolution. However, the naked eye has much better resolution if there is a contrast made available in terms of a color change, or a light emission.

But it is clear from the above discussions that failure rates are very significant, and anyone wishing to prevent an undesirable consequence needs to have an option available. One realization of this concept may be the use of triboluminescent materials.

Triboluminescence is the light generated via the breaking of asymmetrical bonds in a crystal when the material is scratched, crushed, or rubbed (from the Greek tribein, “to rub”). For example, many minerals, such as quartz, glow when scratched; sucrose emits a blue light when crushed; and a diamond may begin to glow while being sawn during the cutting process or when a facet is being ground. Diamonds may fluoresce blue or red. The mechanism of triboluminescence is not fully understood yet, but the research in this area suggests that charge is separated upon fracture of asymmetrical materials and when charges recombine, the electric discharge ionizes the surrounding air, causing a flash of light.

U.S. Pat. No. 4,020,765 describes materials that exhibit triboluminescence, including activated zinc compounds, ZnCdS, zirconium-tin alloys, and CaP₂O₇:Dy.

U.S. Pat. No. 6,071,632 describes triboluminescence occurring in a group of cyclic organic lanthanide compounds.

U.S. Pat. No. 5,905,260 recites a list of organic compounds known to exhibit triboluminescence, including certain europium dibenzoylmethide chelates.

U.S. Pat. No. 6,581,474 describes light indicating assemblies comprising from triboluminescent materials. Suggested uses for damage indicators of the '474 include packing crates, pallets, vault structures, automotive/aircraft components, and building structures. Notably, the invention of '474 relies upon a brittle, porous body to be used as a carrier media for containing a triboluminescent material.

Despite the levels of protection provided by modern protection devices such as condoms, gloves, and food packaging, risks associated with insults to mechanical integrity of these devices still exist, and often users of the device may be unaware of the breakdown of the device's mechanical integrity. For example, a rupture the size of a pinhole in a condom creates an opening sufficient for sperm and/or STDs to travel through, thus potentially leading to undesirable consequences. Such a small hole may not be easily visible to the naked eye, particularly without a thorough, detailed inspection. And, despite the applications of triboluminescence to a variety of mechanical components and structures, known uses involve brittle structures, which are not useful for protection devices, such as condoms, gloves, and food packaging.

### SUMMARY OF THE INVENTION

The present invention relates to non-brittle or flexible materials, preferably polymeric, that are useful in a protective device, such as a condom, glove, or food packaging product, comprising at least one triboluminescent compound.

Non-brittle (and optionally flexible) solid polymeric base materials comprising at least one triboluminescent compound are disclosed.

Protection devices comprising the non-brittle solid polymeric base material disclosed herein are also disclosed.

Additionally, methods of making the non-brittle solid polymeric base materials are provided, said methods comprising incorporating at least one triboluminescent compound into the base material, optionally by admixing the triboluminescent compound with a liquid form of the base material.

Methods of making the protection devices disclosed herein are also provided, said methods comprising forming the protection device from a non-brittle solid polymeric base material, optionally by incorporating the at least one triboluminescent compound into the base material by admixing the triboluminescent compound with a liquid form of the base material to obtain a liquid admixture; applying the liquid admixture to a mold of the detection device; and drying the liquid admixture on the mold to form the protection device.

A condom is also provided comprising at least one triboluminescent compound.

A glove comprising at least one triboluminescent compound is also provided.

A food packaging product comprising at least one triboluminescent compound is also provided.

### BRIEF DESCRIPTION OF THE DRAWINGS

FIG. 1 shows the chemical structure for spiropyran.

FIG. 2 shows the chemical structure for dimethylamino-phenyl-indandione.

FIG. 3 shows the chemical structure for dibenzofuanone.

FIG. 4 shows the chemical structure for polydicetylene.

FIG. 5 shows the chemical structure for piroxicam.

FIG. 6 shows the chemical structure for oxovanadium complex.

### DETAILED DESCRIPTION OF THE INVENTION

In an aspect, the present invention relates generally to non-brittle (and preferably flexible) solid polymeric base materials comprising at least one triboluminescent compound, particularly those that are commonly used to make protective devices, such as condoms, gloves, and plastic films used in packaging products, especially food products.

### Base Materials

As used herein, the phrase “base material” refers to materials commonly used to manufacture protective devices. Such base materials should be flexible, stretchable, and relatively thin. Additionally, such base materials should have sufficient tensile strength and resistance to tearing and should provide bather protection.

Examples of useful base materials include, for example, natural rubber latex, synthetic latex, polyisoprene, polyurethane, vinyl, nitrile rubber, neoprene, polyvinyl chloride, polyvinylidene chloride, low-density polyethylene, an ethylene copolymer, polyester, polystyrene, and a styrene butadiene copolymer.

The base material may also contain additives that improve the physical qualities of the base material. For example, base materials may contain graphene, non-stick agents, surfactants, curing agents, cross-linkers, detackifiers, lubricants, spermicides, vulcanizing accelerators, antioxidants, antibacterial agents, surface modification agents, softening agents, and firming agents.

In the case where the base material is intended to be used in a condom, it is preferably selected from the group consisting of natural rubber latex, synthetic latex, polyurethane, and polyisoprene. Optionally, the base material may further contain graphene, non-stick agents, surfactants, curing agents, cross-linkers, detackifiers, lubricants, and spermicides.

In the case where the base material is intended to be used in a glove, it is preferably selected from the group consisting of natural rubber latex, synthetic latex, nitrile rubber, vinyl, and neoprene. Optionally, the base material may further contain detackifiers, non-stick agents, vulcanizing accelerators, antioxidants, antibacterial agents, and surface modification agents.

In the case where the base material is intended to be used in a packaging, it is preferably selected from the group consisting of polyvinyl chloride, polyvinylidene chloride, low density polyethylene, an ethylene copolymer, polyester, polystyrene, and a styrene butadiene copolymer. Optionally, the base material may further contain additives selected from the group consisting of a plasticizer, additives to increase self-adhesive properties (such as polyisobutene (PIB), and poly[ethylene-vinylacetate] (EVA) copolymer), softening agents, firming agents, and antioxidants.

### Triboluminescent Compounds

In an aspect of the present invention, at least one triboluminescent compound is incorporated into the base material in a manner such that an insult to the mechanical integrity of the base material will result in a color change, owing to action of the triboluminescent compound. Said color change may be reversible, but is preferably permanent.

U.S. Pat. No. 6,581,474 makes specific mention of a number of prior substances used in or mentioned in earlier patents and known to display triboluminscence, including activated zinc compounds (ZnF₂:Mn, ZnS:Ag, ZnS:Mn), ZnCdS, zirconium-tin alloys, and CaP₂O₇:Dy. Also referred to are cyclic organic lanthanide compounds (particularly compounds of Eu, Tb, Dy, and Sm, the atomic numbers of europium, terbium, dysprosium and samarium being, respectively, 63, 65, 66, and 62). Finally, certain europium dibenzoylmethide chelates exhibit intense triboluminescent emission.

Strongest emission is found in europium tetrakis (dibenzoylmethide)-triethylammonium (EuD₄TEA). This emission has the characteristic spectral distribution of the chelated Eu³⁺ ion[20], with decay times of 0.5 msec for the 5₆ 7F₂ lines. Triboluminscence in EuD₄TEA results from excitation of the β-ketoenolate ligand followed by intramolecular energy transfer to the complexed Eu ion. Light output from triboluminscence from EuD₄TEA is one hundred to five hundred times more intense than that from Tb hexaantipyrene triiodide, a previously known efficient triboluminescent material.

In an exemplary embodiment, the triboluminescent compound is selected from the group consisting of activated zinc compounds, ZnCdS, zirconium-tin alloys, CaP₂O₇:Dy, cyclic organic lanthanide compounds, and europium dibenzoylmethide chelates.

In an exemplary embodiment, an activated zinc compound is used, which is preferably selected from the group consisting of ZnF₂:Mn, ZnS:Ag, and ZnS:Mn.

In an exemplary embodiment, a cyclic organic lanthanide compound is used, preferably selected from the group consisting of Eu, Tb, Dy, and Sm.

In an exemplary embodiment, a europium dibenzoylmethide is used, preferably EuD₄TEA.

In an exemplary embodiment, the triboluminescent compound is selected from the group consisting of spiropyran, a dibenzofuranone, a polymeric oxovanadium IV complex carrying a Schiff base ligand, polydicetylene, piroxicam, and an oxovanadium complex.

It should be noted that there are many triboluminescent materials, so those which are the most suitable for the presently disclosed applications may vary from either past use in the literature, or previous patents. In fact nearly sixty years ago, a group of researchers studied about 1700 organic and inorganic substances and found 356 of them to exhibit triboluminscence. It should be noted, however, that the '474 did work with europium tetrakis (dibenzoylmethane)-triethylammonium salt, which has europium in the Eu(III) state, exactly what was found earlier to have the most intense emission.

This fracture response by light emission is one feature sought in the present disclosure. It is presently intended that the triboluminescent material not be embedded in a brittle host but rather have it in embedded in the flexible polymeric material. Upon rupture of the polymer, light emission will occur.

Another acceptable realization is upon rupture, the color of the host material changes from its original color to another stark contrasting color. The idea is that either realization will be immediately recognizable by at least one or both of the partners, alerting them to a compromised situation.

In relation to the need for recognizable light emission or color change, a white-colored transparent device could work with any single wavelength emission in the visible wave lengths, from the red through to the blue regions of the spectrum.

Yet another realization of this concept may be the use of materials displaying permanent color change mechanochromism.

### Permanent Color Change Mechanochromism Materials

There appear to be several mechanically based methods to induce permanent color changes. They are bond breaking, reorganization of crystal packing, and structural phase transition.

### Bond Breaking

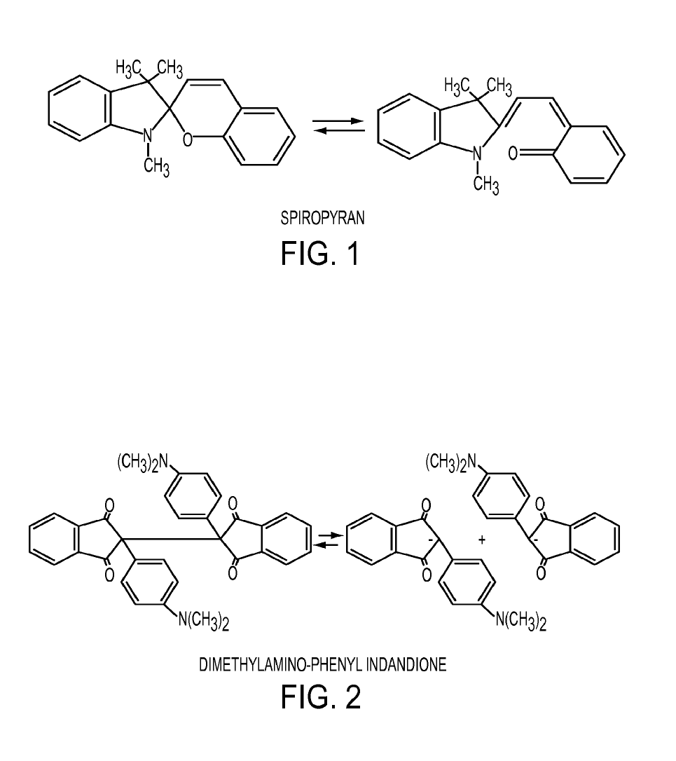

The idea here is to use the breaking of molecular bonds to lead to structural changes, and permanent color changes. For example, color change may be mechanically induced in spiropyran (FIG. 1), by grinding.

Because this color change is reversible, seeking methods to make it permanent is necessary. The color change seems to be based upon simultaneous intramolecular electron transfer and bond disruption, based upon a quantum mechanical calculation. Here mechanochromism is conditioned by formation of defects in the rigid matrix. The spiropyran molecules are excited in the medium of the defects and undergo ring opening to produce the quinoid (colored) molecule, which is trapped in the crystal by lattice forces.

For other molecules, bond breaking generates radicals which cause oscillating coloration. Because permanent color change is sought, so a way to block the color oscillations is desirable. Consider the molecule 2,2-bis[4-(dimethylamino)phenyl]-1,3-indandiones (FIG. 2). It has two forms of radical generated upon bond breaking, with an unpaired electron distributed between the two carbonyls (CH₃)₂N and aromatic rings, resulting in coloration. In the solid phase, the mechanochemical reaction is reversible, which is not desirable. However, onium substituents in the condensed benzene rings (trimethylammonium or 2,4,6-triphenylpyridinium) increase the thermodynamic stability of the radicals, shifting the equilibrium toward the colored products.

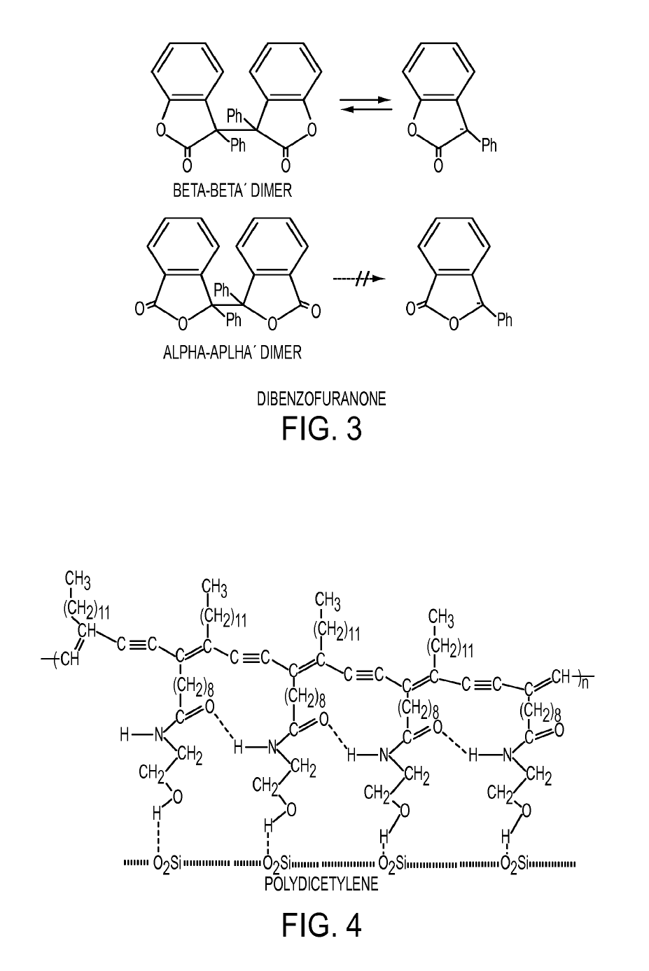

Studies of dibenzofuranones (FIG. 3) showed structural effects on mechanochromism. This molecule has two isomers, an α-α dimer form and a 13-13 dimer form. Reversible homolytic rupture of the β-β bond gives blue colored radicals, which recombine to give the colorless dimer. The β-β isomer experiences strong internal strain and restriction of rotation around the exocyclic bond. Such restriction does not occur of the α-α isomer. To make dibenzofuranones useful, retention of the β-β radicals by stabilization once rupture occurs must be obtained.

A final example is the scission of bonds due to grinding the molecule polymeric oxovanadium IV complexes carrying Schiff base ligands. The orange vanadyl complex with N,N′-disalicylidene-(R,R′)-1,2-diphenyl-1,2-ethanediamine, turns green during grinding.

The compound has a linear chain polymeric structure containing an infinite chain of . . . V═O . . . V═O . . . bonds, and these are cleaved to yield monomer species.

Mechanochromic rearrangement leading to the color change starts at lattice defect sites. Full understanding of the color change process is lacking; however, it may be connected with coordination unsaturation in the monomeric complex formed after grinding.

### Reorganization of Crystal Packing

This process works by slight shifting of the dimensions of the molecules constituting the crystalline form of the organic molecules in a lattice. There is differential motion of the molecules along the coordinate axes, resulting in a slightly different packing arrangement and density.

Consider the thioindigoid molecule, 11-(3′-oxodihydrobenzothiophen-2′ylidene) cyclopenta-[1,2-b:4,3-b′] dibenzothiophene, which undergoes a color change from red to black when powders of it are ground in a mortar with a pestle. Studies imply that mechanical stress initiates partial slipping of the thioindigoid molecules along the stacking axis in the crystal. The interplanar distance is shortened along the molecular stack. A new wide wavelength band appears around 750 nm, causing the color to become black. This color change is interpreted as arising from excitonic interactions between transition dipoles, a result of a reorganized molecular configuration induced by mechanical shearing.

Understanding of the red to black color change in the thioindigoid molecule is based upon experimental data gathered from x-ray diffraction and electron spectroscopy, and from molecular orbital calculations. By keeping the temperature of the transformed organic thioindigoid molecule crystal below 280 C and/or out of organic solvents, this transition in color is permanent. This is no problem because use of the invention is expected at room temperature which is nominally 20 C, with some variance higher or lower for colder or warmer ambient conditions, none of which approach such high temperatures. Likewise, it is not expected that the present invention material be intentionally washed in specialized organic solvents.

### Structural Phase Transition

Shear forces can cause nanoscale mechanochromism of polydicetylene monolayers (FIG. 4) on an atomically flat silicon oxide support. The shear forces change the pendant side chains and are facilitated by defects in the support lattice. Structurally the side chains are pushed toward the surface. The mechanochromism is an irreversible transformation, leading to a color change from blue to red. The initial blue form contains the polymer backbone in the planar all-trans geometry, in which the side chains are in the same plane as the backbone. This geometry permits extended, continuous conjugation between the double and triple bonds of the backbone that runs parallel with the support surface (silicon oxide support).

Shear action leads to rotation about the ordinary carbon-carbon bonds of the polymer backbone, thus altering the backbone planarity. Out-of-plane of orientation of the side chains is achieved, and the conjugation in the backbone is disrupted. The result is a shortened conjugation length. Packing of the alkyl side chain and hydrogen bonding of the head groups jointly restricts the torsional motion of the polymer backbone.

The permanent color change of the polydicetylene is platform based, and the present invention does not have such a platform, except as an overlayer. The interlayer interface would have to provide enough of a disjointed boundary layer to allow the same actions as reported for a structurally rigid silicon platform surface which the polydicetylene sees.

Another possibility is to employ the partially irreversible structural phase transition from the original blue to the red liquid crystal phase of polyacetylene, placed on top of a quartz slide, and induced by stretching or rubbing. Optical micrographs of the polyacetylene coated on glass fibers show different morphology for the blue and red phases. The partially irreversible mechanochromic transition is due to residual strain, and chemical structural factors such as alkyl spacer length. One polyacetylene is the linear crystalline form H (CH₂)ₘ C≡C C≡C (CH₂)₈ [p-C(O)O—C₆H₄—C₆H₄OC(O)-p′] (CH₂)₈ C≡C C≡(CH₂)ₘH. Mechanical action changes the liquid crystal orientation.

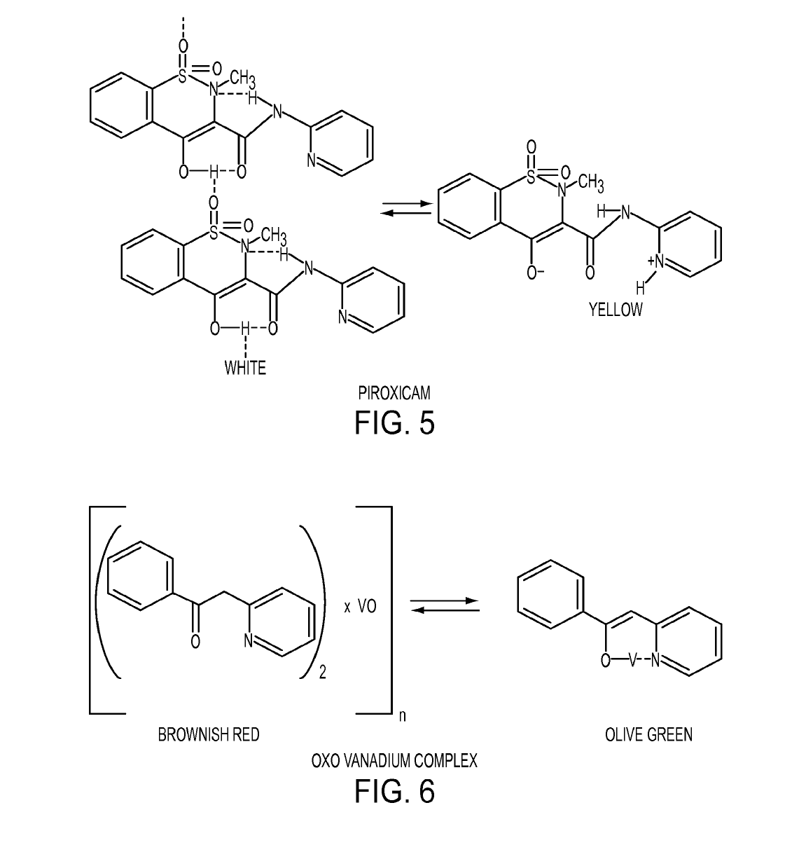

The molecular crystals previously studied can be changed from their crystalline form to an amorphous form by supplying mechanical energy through grinding. This constitutes a phase transformation, and results in the free energy becoming elevated above that of the ordered crystal form. Grinding can disrupt the networks formed at the expense of hydrogen bonding of weak coordination. One of two different molecule crystals which have been studied are chains of piroxicam (FIG. 5) attached ring and partial ring group units with sulfur S, nitrogen N atomic substitutes for the carbon C ring atoms. Double bonds exist of off the S atom, which attach one unit to the next unit's partial ring H atom. It is this weak bond which is broken upon mechanical action, changing the initial chain molecular white color to yellow.

The result is that the ground crystal becomes an amorphous state consisting entirely of these single unit monomers. The ground monomer products do have a high propensity to revert back to the crystalline form, but this tendency may be substantially thwarted if the crystals are embedded in our host organic condom matrix. There may be other ways to stop the release of free energy and return to the original crystalline state, having to do with better understanding of its thermal dependence. The conclusion is that with proper embedding of the crystal in the host matrix organic material, permanent color change upon puncture could occur.

A second form of molecule, an oxovanadium complex (FIG. 6), going from a crystalline form to an amorphous form by supplying mechanical energy through grinding, is seen in repeated double molecular units of closed and open carbon rings constituting a triad, coordinated with vanadium oxide VO. Representing the molecule as [(Triad)₂ VO]ₙ in the crystalline state, mechanical action reduces it to the monomer unit with the vanadium atom V linking the middle open ring in the triad, becoming a closed ring upon creation of an O—V—N bond. Color is changed from brownish red to olive green when going from the crystalline to amorphous form.

Methods of Incorporating Triboluminescent Compounds with the Base Materials

Triboluminescent compounds can be embedded in any carrying matrix material in an inactive sense by any standard mixing process which may include use of particles or strings of the triboluminescent compounds, such that they will experience mechanical insults caused by tears and rips and punctures to the fabric. It is in an inactive sense in that there is not chemical attachment of the triboluminescent compounds to the matrix chemical material.

However, it is possible in a second realization, that the triboluminescent compounds are attached to the matrix chemicals in a way to merely carry them along, not affecting their basic properties.

Either realization would allow proper action of the triboluminescent compounds action.

### Protection Devices

In an aspect, protection devices are disclosed incorporating triboluminescent compounds in a manner that an insult to the mechanical integrity of the base material will result in a color change, owing to action of the triboluminescent compound. Said color change may be reversible, but is preferably permanent. For example, the protection devices may be made from the foregoing non-brittle solid polymeric base materials.

As used herein, the term “protection device” shall refer to devices that provide a barrier between an object and the surrounding atmosphere.

Examples of protection devices include condoms, gloves (such as surgical or exam gloves), and plastic films used in packaging products (including, for example plastic wrap—also referred to as cling wrap or food wrap).

Such protection devices, like the base materials from which they are made, should be flexible, stretchable, and relatively thin. Additionally, such protection devices should have sufficient tensile strength and resistance to tearing and should provide barrier protection.

In an exemplary embodiment, the protection devices are made by a method comprising incorporating the at least one triboluminescent compound into the base material by admixing the triboluminescent compound with a liquid form of the base material to obtain a liquid admixture; applying the liquid admixture to a mold of the detection device; and drying the liquid admixture on the mold to form the protection device.

1. A non-brittle solid polymeric base material comprising at least one triboluminescent compound.

2. The non-brittle solid polymeric base material of claim 1, wherein the base material is selected from the group consisting of natural rubber latex, synthetic latex, polyisoprene, polyurethane, vinyl, nitrile rubber, and neoprene.

3. The non-brittle solid polymeric base material of claim 1, wherein the triboluminescent compound is selected from the group consisting of activated zinc compounds, ZnCdS, zirconium-tin alloys, CaP₂O₇: Dy, cyclic organic lanthanide compounds, and europium dibenzoylmethide chelates.

4. The non-brittle solid polymeric base material of claim 3, wherein the activated zinc compound is selected from the group consisting of ZnF₂:Mn, ZnS:Ag, and ZnS:Mn.

5. The non-brittle solid polymeric base material of claim 3, wherein the cyclic organic lanthanide compounds is selected from the group consisting of Eu, Tb, Dy, and Sm.

6. The non-brittle solid polymeric base material of claim 3, wherein the europium dibenzoylmethide is EuD4TEA.

7. The non-brittle solid polymeric base material of claim 1, wherein the at least one triboluminescent compound is selected from the group consisting of spiropyran, a dibenzofuranone, a polymeric oxovanadium IV complex carrying a Schiff base ligand, polydicetylene, piroxicam, and an oxovanadium complex.

8. A protection device comprising the non-brittle solid polymeric base material of claim 1.

9. The protection device of claim 8, wherein the protection device is selected from the group consisting of a condom, a glove, and a food packaging product.

10. A method of making the non-brittle solid polymeric base material of claim 1, said method comprising incorporating the at least one triboluminescent compound into the base material.

11. The method of claim 10, wherein the tiiboluminescent compound is admixed with a liquid form of the base material.

12. A method of making a protection device of claim 8, said method comprising forming the protection device from said non-brittle solid polymeric base material.

13. The method of claim 12 comprising:
a) incorporating the at least one triboluminescent compound into the base material by admixing the triboluminescent compound with a liquid form of the base material to obtain a liquid admixture;
b) applying the liquid admixture to a mold of the detection device; and
c) drying the liquid admixture on the mold to form the protection device.

14. A condom comprising at least one triboluminescent compound.

15. The condom of claim 14, wherein the condom is made from a non-brittle solid polymeric base material selected from the group consisting of natural robber latex, synthetic latex, polyisoprene, polyurethane, vinyl, nitrile rubber, and neoprene.

16. The condom of claim 14, wherein the triboluminescent compound is selected from the group consisting of activated zinc compounds, ZnCdS, zirconium-tin alloys, CaP₂O₇:Dy, cyclic organic lanthanide compounds, and europium dibenzoylmethide chelates.

17. The condom of claim 16, wherein the activated zinc compound is selected from the group consisting of ZnF₂:Mn, ZnS:Ag, and ZnS:Mn.

18. The condom of claim 16, wherein the cyclic organic lanthanide compounds is selected from the group consisting of Eu, Tb, Dy, and Sm.

19. The condom of claim 16, wherein the europium dibenzoylmethide is EuD₄TEA.

20. The condom of claim 14, wherein the at least one triboluminescent compound is selected from the group consisting of spiropyran, a dibenzofuranone, a polymeric oxovanadium IV complex carrying a Schiff base ligand, polydicetylene, piroxicam, and an oxovanadium complex.

21. A glove comprising at least one triboluminescent compound.

22. The glove of claim 21, wherein the glove is made from a non-brittle solid polymeric base material selected from the group consisting of natural rubber latex, synthetic latex, polyisoprene, polyurethane, vinyl, nitiile rubber, and neoprene.

23. The glove of claim 21, wherein the triboluminescent compound is selected from the group consisting of activated zinc compounds, ZnCdS, zirconium-tin alloys, CaP₂O₇:Dy, cyclic organic lanthanide compounds, and europium dibenzoylmethide chelates.

24. The glove of claim 23, wherein the activated zinc compound is selected from the group consisting of ZnF₂:Mn, ZnS:Ag, and ZnS:Mn.

25. The glove of claim 23, wherein the cyclic organic lanthanide compounds is selected from the group consisting of Eu, Tb, Dy, and Sm.

26. The glove of claim 23, wherein the europium dibenzoylmethide is EuD₄TEA.

27. The glove of claim 21, wherein the at least one triboluminescent compound is selected from the group consisting of spiropyran, a dibenzofuranone, a polymeric oxovanadium IV complex carrying a Schiff base ligand, polydicetylene, piroxicam, and an oxovanadium complex.

28. A food packaging product comprising at least one triboluminescent compound.

29. The food packaging product of claim 28, wherein the food packaging product is made from a non-brittle solid polymeric base material selected from the group consisting of natural rubber latex, synthetic latex, polyisoprene, polyurethane, vinyl, nitiile rubber, and neoprene.

30. The food packaging product of claim 28, wherein the triboluminescent compound is selected from the group consisting of activated zinc compounds, ZnCdS, zirconium-tin alloys, CaP₂O₇:Dy, cyclic organic lanthanide compounds, and europium dibenzoylmethide chelates.

31. The food packaging product of claim 30, wherein the activated zinc compound is selected from the group consisting of ZnF₂:Mn, ZnS:Ag, and ZnS:Mn.

32. The food packaging product of claim 30, wherein the cyclic organic lanthanide compounds is selected from the group consisting of Eu, Tb, Dy, and Sm.

33. The food packaging product of claim 30, wherein the europium dibenzoylmethide is EuD₄TEA.

34. The food packaging product of claim 28, wherein the at least one triboluminescent compound is selected from the group consisting of spiropyran, a dibenzofuranone, a polymeric oxovanadium IV complex carrying a Schiff base ligand, polydicetylene, piroxicam, and an oxovanadium complex.
